(19) United States
(12) Patent Application Publication
(10) Pub. No.: US 2007/0190187 A1
(43) Pub. Date: Aug. 16, 2007

(54) FORMULATION FOR ENHANCED DELIVERY OF PHENETHYLAMINE

(76) Inventors: Bruce W. Kneller, Randolph, MA (US); Richard S. Gaspari, Jackson, NJ (US)

Correspondence Address: LAW OFFICE OF STEVEN B. LEAVITT, L.L.P., P.O. BOX 537, ROWLETT, TX, 75030-0537, US

(21) Appl. No.: 11/737,138

(22) Filed: Apr. 18, 2007

Related U.S. Application Data
(60) Provisional application No. 60/824,811, filed on Sep. 7, 2006.

(51) Int. Cl. A61K 36/889; A01N 33/02; A61K 31/135; A61K 36/254; A61K 36/38; A61K 36/84; A61K 36/67; A61K 36/28; A61K 36/258

(52) U.S. Cl. 424/725; 424/727; 424/728; 424/730; 424/733; 424/734; 424/737; 424/746; 424/749; 424/752; 424/778; 514/653; 560/40; 514/317; 514/469; 514/283

(57) ABSTRACT

The present invention is directed to a formulation comprising phenethylamine or a derivative thereof, and an MAO-B enzyme inhibitor. The present invention is also directed to a method of increasing the absorption of phenethylamine or a derivative thereof, into the bloodstream, cells and tissue. The method includes administering phenethylamine or a derivative thereof, in combination with an MAO-B enzyme inhibitor.

### BACKGROUND OF THE INVENTION

1. Technical Field

The present invention relates to a formulation comprising phenethylamine or a derivative thereof, and a Monoamine Oxidase Type-B Enzyme Inhibitor commonly refered to as MAO-B enzyme inhibitor or MAO-Bi. The formulation may be used in dietary products and pharmaceuticals.

2. Description of Related Art

Phenethylamine (β-Phenylethylamine, CAS# 64-04-0) is a plant alkaloid and monoamine. In the human brain, it is believed to function as a neuromodulator or neurotransmitter, meaning it is a trace amine. Phenethylamine is a colorless liquid that forms a solid carbonate salt with carbon dioxide (CO₂) upon exposure to air, and in nature is synthesized from the amino acid phenylalanine by enzymatic decarboxylation. Phenethylamine can also be synthesized from the amino acids tyrosine and phenylalanine in vivo. Phenethylamine and is found in many foods, especially after microbial fermentation, for example, in chocolate. It has been suggested that phenethylamine from food such as chocolate may have psychoactive effects in sufficient quantities. However, it is quickly metabolized by the enzyme Monoamine Oxidase Type-B Enzyme Inhibitor commonly referred to as MAO-B enzyme inhibitor or MAO-Bi. The quick metabolizing of phenethylamine prevents significant of phenethylamine concentrations from reaching the brain for any length of time and imparting a neurological or psychological effect unless adminstered in exceptionally high and inconvenient doses.

Phenethylamine is also found in trace amounts in foodstuffs/supplements, such as Blue Green Algae, a phytoplankton also known as marine microalgae, which are single cell plants that are the “vegetation” of the ocean. Phytoplankton are responsible for making up to 90% of the Earth's oxygen and with the ability to convert sunlight, warmth, water, and minerals into proteins, carbohydrates, vitamins and amino acids, they are considered to be the basis of all other life forms on earth. Phytoplankton are also sometimes found in processed meats such as bologna and salami.

In the human brain, phenethylamine is thought to induce its effects by having a greater affinity for the re-uptake mechanism for dopamine in presynaptic vesicles. When present in the brain, phenethylamine is “locked into” the presynaptic vesicles and takes up the same space normally taken by the neurotransmitter dopamine. This process may lead to an increase in free-circulating, available dopamine in the presynaptic terminal and a higher concentration of dopamine diffusing into the synaptic cleft, therefore enhancing dopaminergic transmission. The ability of phenethylamine to effect dopaminergic transmission gives some interesting psychopharmacological characteristics such as increasing ability to concentrate and elevation of mood.

Phenethylamine has also been demonstrated to effect and increase other neurotransmitter's transmission in the brain, such as norepinephrine. Norepinephrine is also believed to be an integral factor in allowing humans to experience happiness or euphoria and decreasing appetite. For example, in at least one study, if an animal is implanted with an electrode in an area of the brain concentrated in norepinephrine, and the electrode is activated by a pedal that the animal has access to, the animal will disregard food and water and will press the pedal relentlessly until exhaustion to elicit an electrical impulse in the norepinephrine concentrated area of the brain.

It has been suggested that phenethylamine found in food, such as chocolate, may have psychoactive effects in sufficient quantities. However, the phenethylamine found in food it is quickly metabolized by the enzyme MAO-B, thus preventing significant concentrations from reaching the brain for any significant length of time and imparting a neurological or psychological effect unless administered in exceptionally high and inconvenient doses.

What is needed is a method or compound to inhibit the effects of the enzyme MAO-B and would allow much more phenylethylamine to reach the brain per dose administered. It would be beneficial if the inhibitor of the enzyme MAO-B were to be a DSHEA compliant foodstuff/supplement that would allow the combination of phenylethylamine and the MAO-B enzyme inhibitor to be sold as a food supplement. It would be even more beneficial if the inhibitor consisted of an oral dose ranging between 160 mg and 1000 mg per dose with one or more naturally occurring, DSHEA compliant inhibitors of the enzyme, MAO-B.

### SUMMARY OF INVENTION

The present invention solves the above-described problem by providing a formulation comprising phenethylamine or a derivative thereof, and a Monoamine Oxidase Type-B Enzyme Inhibitor commonly referred to as MAO-B enzyme inhibitor or MAO-Bi enzyme inhibitor. The formulation of the present invention provides enhanced absorption and delayed metabolism of phenethylamine, or a derivative thereof, into the bloodstream, cells and tissue.

More particularly, the formulation of the present invention may be a pharmaceutical, a vaccine, a topical or transdermal formulation or a foodstuff such as a dietary supplement. The formulation preferably comprises between about 10% and about 75% of phenethylamine or a derivative thereof. In addition, the formulation preferably comprises between about 10% and about 50% of an MAO-B enzyme inhibitor.

Another aspect of the present invention is directed to a method of increasing the absorption and delaying the metabolism of phenethylamine or a derivative thereof, into the bloodstream, cells and tissue. The method includes administering phenethylamine or a derivative thereof, in combination with an MAO-B enzyme inhibitor.

More particularly, the phenethylamine or a derivative thereof, and the MAO-B enzyme inhibitor are administered in a formulation. The formulation may be a pharmaceutical, a vaccine, a topical or transdermal compound or a foodstuff such as a dietary supplement.

Other features and advantages of the present invention will become apparent upon reading the following detailed description of embodiments of the invention, when taken in conjunction with the appended claims.

### DETAILED DESCRIPTION OF THE INVENTION

Substituted phenethylamines are a broad and diverse class of compounds that include neurotransmitters, hormones, stimulants, hallucinogens, entactogens, anorectics, bronchodilators, and antidepressants. The phenethylamine structure can also be found as part of more complex ring systems such as the ergoline system of LSD or the morphinan system of morphine. Substituted phenethylamines carry additional chemical modifications at the phenyl ring, the sidechain, or the amino group. Amphetamines are homologues of phenethylamines carrying an alpha-methyl (α-CH₃) group at the sidechain carbon atom next to the amino group. Catecholamines are phenethylamines carrying two hydroxy groups in positions 3 and 4 of the phenyl ring. Examples are the hormones and neurotransmitters dopamine, epinephrine (adrenaline), and norepinephrine (noradrenaline). The aromatic amino acids phenylalanine and tyrosine are phenethylamines carrying a carboxyl group (COOH) in the alpha position.

Many substituted phenethylamines are pharmacologically active drugs due to their similarity to the monoamine neurotransmitters. Various classes of pharmacologically active substituted phenethylamines include, but are not limited to: stimulants, like the plant alkaloids ephedrine and cathinone and the synthetic drugs amphetamine (speed, benzedrine) and methylphenidate; hallucinogens, such as plant alkaloid mescaline and the synthetic drug 2C-B; empathogen-entactogens like MDMA (ecstasy) and MDA; anorectics, such as phentermine, fenfluramine, and amphetamine; bronchodilators like salbutamol and ephedrine; and antidepressants, like bupropion and the monoamine oxidase inhibitors phenelzine and tranylcypromine.

Below is the structure of Phenylethylamine.

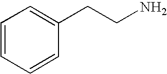

Following is the general structure of phenethylamines and amphetamines (see Table 1 below).

TABLE 1

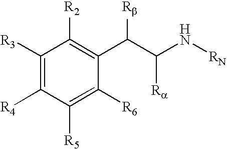

Substituted phenethylamines, tabulated by structure

| Short Name | R_{α} | R_{β} | R₂ | R₃ | R₄ | R₅ | R_{N} | Full Name |
|---|---|---|---|---|---|---|---|---|
| Tyramine | | | | | OH | | | 4-hydroxy- |
| | | | | | | | | phenethylamine |
| Dopamine | | | | OH | OH | | | 3,4-dihydroxy- |
| | | | | | | | | phenethylamine |
| Epinephrine | | OH | | OH | OH | | CH₃ | β,3,4-trihydroxy-N- |
| (Adrenaline) | | | | | | | | methyl-phenethylamine |
| Norepinephrine | | OH | | OH | OH | | | β,3,4- |
| (Noradrenaline) | | | | | | | | trihydroxyphenethylamine |
| Salbutamol | | OH | | | OH | CH₂CH₂OH | C(CH₃)₃ | 4-(2-(tert-Butylamino) |
| | | | | | | | | 1-hydroxyethyl)-2- |
| | | | | | | | | (hydroxymethyl)phenol |
| Amphetamine | CH₃ | | | | | | | α-methyl- |
| | | | | | | | | phenethylamine |
| Methamphetamine | CH₃ | | | | | | CH₃ | N-methyl-amphetamine |
| Levmetamfetamine | CH₃ | | | | | | CH₃ | N-methyl-amphetamine |
| Ephedrine, | CH₃ | OH | | | | | CH₃ | N-methyl-β-hydroxy- |
| pseudoephedrine | | | | | | | | amphetamine |
| Cathine | CH₃ | OH | | | | | | β-hydroxy- |
| | | | | | | | | amphetamine |
| Cathinone | CH₃ | ═O | | | | | | β-keto-amphetamine |
| Methcathinone | CH₃ | ═O | | | | | CH₃ | N-methyl-β-keto- |
| | | | | | | | | amphetamine |
| Bupropion | CH₃ | ═O | | Cl | | | C(CH₃)₃ | 3-chloro-N-tert-butyl-β- |
| | | | | | | | | keto amphetamine |
| Fenfluramine | CH₃ | | | CF₃ | | | CH₂CH | 3-trifluoromethyl-N- |
| | | | | | | | | ethyl-amphetamine |
| Phentermine | CH₃, CH₃ | | | | | | | α,α-dimethyl- |
| | | | | | | | | phenethylamine |
| Mescaline | | | | OCH₃ | OCH₃ | OCH₃ | | 3,4,5-trimethoxy- |
| | | | | | | | | phenethylamine |

| | | | | |
|---|---|---|---|---|
| MDA | CH₃ | —O—CH₂—O— | | 3,4-methylenedioxy- |
| | | | | amphetamine |
| MDMA | CH₃ | —O—CH₂—O— | CH₃ | 3,4-methylenedioxy-N- |
| | | | | methyl-amphetamine |

As explained above, phenethylamine is quickly metabolized by the enzyme Monoamine Oxidase Type-B Enzyme Inhibitor commonly referred to as MAO-B enzyme inhibitor or MAO-Bi, thereby preventing significant concentrations of phenethylamine from reaching the brain and other organs for a sufficient period of time to elucidate the desired effect(s).

In one test, phenylethylamine was co-administered with a moiety/moieties that inhibited the effects of the enzyme MAO-B, and as a result, an increased amount of phenethylamine or a derivative thereof, is absorbed into the bloodstream, cells and tissue and accordingly, a greater amount of phenethylamine or a derivative thereof, is allowed to reach the brain for a greater period of time per dose administered before it is metabolized into an psycho-pharmacologically inert substance. Therefore, the combination of phenylethylamine or derivative thereof and the MAO-B enzyme inhibitor may be formulated as a dietary supplement, other foodstuff, a pharmaceutical, a topical or transdermal compound or a vaccine. The Dietary Supplement Health and Education Act of 1994, commonly referred to as DSHEA, defines “dietary supplement” as any product (except tobacco) that contains at least one of the following: (1) a vitamin, (2) a mineral, (3) an herb or botanical, (4) an amino acid, (5) a dietary substance “for use to supplement the diet by increasing total dietary intake,” or (6) any concentrate, metabolite, constituent, extract, or combination of any of the aforementioned ingredients. Examples of dietary supplements include, but are not limited to nutritional products; sports performance products, including ergogenic sports performing products; weight loss products; and meal replacement products. Dietary supplements may exist in various forms, including, but not limited to tablets, capsules, caplets, powders, drinks including shakes, and solid food items including snack bars, etc. For purposes of the present invention, a foodstuff is defined as a substance that can be used or prepared for use as food or extracted from food.

The pharmaceutical compositions of this invention can be administered to an individual orally, rectally, parenterally, intracistemally, intravaginally, intraperitoneally, topically (as by powders, ointments, or drops), supralingually (on the tongue) sublingually (under the tongue), bucally (held in the buccal pouch), as an oral or nasal spray, as a chewing gum, or any other method known in the art for delivering a similar pharmaceutical composition. The oral spray may be in the form of a powder or mist which is delivered to the deep lungs by oral inhalation. Liquid dosage forms for oral administration include pharmaceutically acceptable emulsions, microemulsions, solutions, suspensions, syrups and elixirs.

There exists many naturally occurring, DSHEA compliant inhibitors of MAO-B which may be used in the formulation of the present invention, including but not limited to: Piper longum and Piper methisticum Kava (including but not limited to the pure moieties contained in Piper longum and/or Piper methisticum Kava: piperine, desmethoxyyangonin, (+/−)-methysticin, yangonin, (+/−)-dihydromethysticin, (+/−)- dihydrokavain, (+/−)-kavain.), Ginkgo biloba (including but not limited to the pure moieties contained in Ginko biloba: kaempferol and isorhamnetin), Hypericum perforatum L (aka—St. John's wort including but not limited to the pure moieties contained in Hypericum perforatum: quercetin and hypericin), other Hypericum species including, Hypericum caprifoliatum, Hypericum carinatum, Hypericum connatum, Hypericum cordatum, Hypericum myrianthum, Hypericum piriai, Hypericum polyanthemum and Hypericum brasiliense (including but not limited to the pure moieties contained in some or all species of Hypericum: 6-isobutyryl-5,7-dimethoxy-2,2-dimethylbenzopyran, 7-hydroxy-6-isobutyryl-5-methoxy-2,2-dimethylbenzopyran, 5-hydroxy-6-isobutyryl-7-methoxy-2,2dimethylbenzopyran), Evodia rutaecarpa (including but not limited to the following moieties found in Evodia rutaecarpa: 1-methyl-2-undecyl-4(1H)-quinolone, evodiamine, rutaecarpine, limonin, and goshuyuamide II), Rhizoma acori gramine (including but not limited to the following moieties found in Rhizoma acori gramine: eugenol and beta-asarone), Sinofranchetia chinensis (including but not limited to the following moieties found in Sinofranchetia chinensis: liquiritigenin and isoliquiritigenin), Uncaria rhynchophylla (including but not limited to the following moieties found in Uncaria rhynchophylla: (+)-catechin and (−)-epicatechin.), Salvia divinorum (including but not limited to the following moiety found in Salvia divinorum: salvinorin A), Bacopa monniera (including but not limited to the following moiety found in Bacopa monniera: Bacoside A), Cudrania tricuspidata (including but not limited to the following moieties found in Cudrania tricuspidata: Gancaonin A, 4′-O-methylalpinumisoflavone, and alpinumisoflavone), Lithospermum erythrorhizon (including but not limited to the following moieties found in Lithospermum erythrorhizon: acetylshikonin, shikonin, and shikonofuran E), Sophora flavescens (including but not limited to the following moieties found in Sophora flavescens: formononetin, kushenol F, oxymatrine, trifolirhizin, and beta-sitosterol), Melastoma candidum (including but not limited to the following moieties found in Melastoma candidum: quercitrin, isoquercitrin and rutin), Coptis chinensis (including but not limited to the following moieties found in Coptis chinensis: jatrorrhizine, berberine and palmatine), Opuntia ficus-indica (including but not limited to the following moieties found in Opuntia ficus-indic: 1-monomethyl citrate, 1,3-dimethyl citrate, trimethyl citrate and 1-methyl malate, Uncaria tomentosa, Uncaria gambir, Radix Paeoniae Alba, Green tea (including but not limited to the following moieties found in Green tea (−)-epicatechin, (−)-epicatechin-3-gallate, (−)-epigallocatechin and (−)-epigallocatechin-3-gallate), various types of yams (Dioscorea species—including but not limited to the following moieties found in various yam species: diosgenin, 5-keto-diosgenin, 6-keto-diosgenin) as well as the pure moiety/supplement hordenine (N,N-dimethyltyramine) which is found in barley and grass.

In one embodiment, the formulation is administered in a dose comprising between about 160 mg and about 1000 mg of phenylethylamine or a derivative thereof in combination with one or more naturally occurring, DSHEA compliant inhibitors of the enzyme, MAO-B.

In one embodiment, the formulation of the present invention comprises between about 10% and about 50% of an MAO-B enzyme inhibitor. More preferably, the formulation of the present invention comprises between about 33% and about 40% of an MAO-B enzyme inhibitor.

Furthermore, the formulation of one embodiment comprises between approximately 10% and approximately 75% of phenethylamine or a derivative thereof. Preferably, the formulation comprises between approximately 25% and approximately 50% of phenethylamine or a derivative thereof.

In one embodiment, the formulation is administered in a dose comprising between approximately 160 mg and approximately 1000 mg of phenethylamine or a derivative thereof. Preferably, the formulation is administered in a dose comprising between approximately 250 mg and approximately 500 mg of phenethylamine or a derivative thereof.

The present invention is also related to a method of increasing the absorption of and delaying the metabolism of phenethylamine or a derivative thereof, into/by the bloodstream, cells and tissue. For example, the method of the present invention allows an increased amount of phenethylamine or a derivative thereof, to reach the brain per dose administered.

The method of the present invention includes administering phenethylamine or a derivative thereof in combination with an MAO-B enzyme inhibitor. The phenethylamine or derivative thereof, and the MAO-B enzyme inhibitor may be administered by any method known to those having skill in the art, including but not limited to being administered orally or parenterally. The phenethylamine or derivative thereof, is administered in a dose between approximately 160 mg and approximately 1000 mg. Preferably, the phenethylamine or derivative thereof is administered in a dose between approximately 250 mg and approximately 500 mg.

In one embodiment, the formulation used in the method of the present invention comprises between about 10% and about 50% of an MAO-B enzyme inhibitor. Preferably, the formulation comprises between about 33% and about 40% of an MAO-B enzyme inhibitor.

Furthermore, the formulation used comprises between approximately 10% and approximately 75% of phenethylamine or a derivative thereof. Preferably, the formulation comprises between approximately 25% and approximately 50% of phenethylamine or a derivative thereof.

The formulations and method of the present invention, utilizing DSHEA compliant compounds, provide for less frequent and lower oral dosing of phenethylamine or a derivative thereof, to achieve desired effect(s). Desired effects of the formulation and method of the present invention, include but are not limited to: decreased appetite; increased alertness and mental clarity; improved mood; increased basal metabolic rate; reversal of depressive episodes; relief of menstrual cramps and negative conditions associated with menstruation; increased bronchodilation; amelioration of hypersomnia or narcoleptic conditions; increase attention span; alleviation of pain, specifically, but not limited to, headache and backache; increased lipolysis and inhibition of lipogenesis; fat loss; and/or improvement or enhancement of athletic performance. Accordingly, the formulations and method of the present invention have been proven effective to treat various illnesses and conditions including, but not limited to obesity, depression, bronchodilation, hypersomnia, and narcolepsy.

It should also be understood that the foregoing relates to exemplary embodiments of the invention and that modifications may be made without departing from the spirit and scope of the invention as set forth in the following claims.

What is claimed is:

1. A pharmaceutical composition for inhibiting the effects of the enzyme MAO-B wherein the pharmaceutical composition is a DSHEA compliant inhibitor of MAO-B and allows phenylethylamine to reach the brain in sufficient quantities to elucidate a predetermined effect.

2. The pharmaceutical composition according to claim 1, wherein the DSHEA compliant inhibitor wherein the inhibitor is selected from the group of Piper longum and Piper methisticum Kava (including but not limited to the pure moieties contained in Piper longum and/or Piper methisticum Kava: piperine, desmethoxyyangonin, (+/−)-methysticin, yangonin, (+/−)-dihydromethysticin, (+/−)-dihydrokavain, (+/−)-kavain.), Ginkgo biloba (including but not limited to the pure moieties contained in Ginko biloba: kaempferol and isorhamnetin), Hypericum perforatum L (aka—St. John's wort including but not limited to the pure moieties contained in Hypericum perforatum: quercetin and hypericin), other Hypericum species including, Hypericum caprifoliatum, Hypericum carinatum, Hypericum connatum, Hypericum cordatum, Hypericum myrianthum, Hypericum piriai, Hypericum polyanthemum and Hypericum brasiliense (including but not limited to the pure moieties contained in some or all species of Hypericum: 6-isobutyryl-5,7-dimethoxy-2,2-dimethylbenzopyran, 7-hydroxy-6-isobutyryl-5-methoxy-2,2-dimethylbenzopyran, 5-hydroxy-6-isobutyryl-7-methoxy-2,2dimethylbenzopyran), Evodia rutaecarpa (including but not limited to the following moieties found in Evodia rutaecarpa: 1-methyl-2-undecyl-4(1H)-quinolone, evodiamine, rutaecarpine, limonin, and goshuyuamide II), Rhizoma acori gramine (including but not limited to the following moieties found in Rhizoma acori gramine: eugenol and beta-asarone), Sinofranchetia chinensis (including but not limited to the following moieties found in Sinofranchetia chinensis: liquiritigenin and isoliquiritigenin), Uncaria rhynchophylla (including but not limited to the following moieties found in Uncaria rhynchophylla: (+)-catechin and (−)-epicatechin.), Salvia divinorum (including but not limited to the following moiety found in Salvia divinorum: salvinorin A), Bacopa monniera (including but not limited to the following moiety found in Bacopa monniera: Bacoside A), Cudrania tricuspidata (including but not limited to the following moieties found in Cudrania tricuspidata: Gancaonin A, 4′-O-methylalpinumisoflavone, and alpinumisoflavone), Lithospermum erythrorhizon (including but not limited to the following moieties found in Lithospermum erythrorhizon: acetylshikonin, shikonin, and shikonofuran E), Sophora flavescens (including but not limited to the following moieties found in Sophora flavescens: formononetin, kushenol F, oxymatrine, trifolirhizin, and beta-sitosterol), Melastoma candidum (including but not limited to the following moieties found in Melastoma candidum: quercitrin, isoquercitrin and rutin), Coptis chinensis (including but not limited to the following moieties found in Coptis chinensis: jatrorrhizine, berberine and palmatine), Opuntia ficus-indica (including but not limited to the following moieties found in Opuntia ficus-indic: 1-monomethyl citrate, 1,3-dimethyl citrate, trimethyl citrate and 1-methyl malate, Uncaria tomentosa, Uncaria gambir, Radix Paeoniae Alba, Green tea (including but not limited to the following moieties found in Green tea (−)-epicatechin, (−)-epicatechin-3-gallate, (−)-epigallocatechin and (−)-epigallocatechin-3-gallate), various types of yams (Dioscorea species—including but not limited to the following moieties found in various yam species: diosgenin, 5-keto-diosgenin, 6-keto-diosgenin) or the pure moiety/supplement hordenine (N,N-dimethyltyramine) which is found in barley and grasses).

3. The pharmaceutical composition according to claim 2, wherein the inhibitor is administered in a dose comprising between approximately 160 mg and approximately 1000 mg of phenylethylamine or a derivative thereof in combination with one or more DSHEA compliant inhibitors.

4. The formulation of claim 1, wherein the formulation comprises between about 10% and about 75% of phenethylamine or a derivative thereof.

6. The formulation of claim 1, wherein the formulation comprises between about 10% and about 50% of an MAO-B enzyme inhibitor.

7. The formulation of claim 6, wherein the formulation comprises between about 33% and about 40% of an MAO-B enzyme inhibitor.

8. The formulation of claim 1, wherein the formulation is administered in a dose comprising between approximately 160 mg and approximately 1000 mg of phenethylamine or a derivative thereof.

9. The formulation of claim 1, wherein the MAO-B enzyme inhibitor is Piper longum, Ginkgo biloba, Hypericum perforatum L, Evodia rutaecarpa, Piper methysticum Forster, or N,N-dimethyltyramine.

10. A method of increasing the absorption and delaying the metabolism of phenethylamine into/by the bloodstream, cells and tissue comprising administering phenethylamine or a derivative thereof in combination with an MAO-B enzyme inhibitor.

11. The method of claim 10, wherein the phenethylamine or derivative thereof and the MAO-B enzyme inhibitor are administered in a formulation.

12. The method of claim 11, wherein the formulation is a foodstuff, a pharmaceutical, or a vaccine.

13. The method of claim 12, wherein the formulation is a dietary supplement.

14. The method of claim 11, wherein the formulation comprises between about 10% and about 75% of phenethylamine or a derivative thereof.

15. The method of claim 11, wherein the formulation comprises between about 33% and about 40% of an MAO-B enzyme inhibitor.

16. The method of claim 10, wherein the phenethylamine or derivative thereof is administered in a dose between approximately 160 mg and approximately 1000 mg.

17. The method of claim 16, wherein the phenethylamine or derivative thereof is administered in a dose between approximately 250 mg and approximately 500 mg.

18. The method of claim 10, wherein the administration of phenethylamine or derivative thereof in combination with the MAO-B enzyme inhibitor decreases appetite, increases alertness and mental clarity, improves mood, enhances athletic performance, induces and sustains fat loss or increases basal metabolic rate.

19. The method of claim 10, wherein the administration of phenethylamine or derivative thereof in combination with the MAO-B enzyme is used to treat obesity, depression, bronchodilation, hypersomnia, or narcolepsy.

20. The method of claim 10, wherein the phenethylamine or derivative thereof and the MAO-B enzyme inhibitor are administered orally or parenterally.
